(19) United States
(12) Patent Application Publication
(10) Pub. No.: US 2023/0147583 A1
(43) Pub. Date: May 11, 2023

(54) Protective Breathing Apparatus For Viruses And Toxic Particles

(71) Applicant: Peter Sing, McCleary, WA (US)

(72) Inventor: Peter Sing, McCleary, WA (US)

(21) Appl. No.: 17/523,700

(22) Filed: Nov. 10, 2021

(51) Int. Cl. A62B 7/10; A62B 18/00; A62B 18/10; A62B 18/04; A61L 9/20; A61L 9/14; A42B 1/017

(52) U.S. Cl. CPC A62B 7/10; A62B 18/003; A62B 18/10; A62B 18/045; A61L 9/20; A61L 9/145; A42B 1/017; A61L 2209/14; A61L 2209/22; A61L 2209/134; A61L 2209/12

(57) ABSTRACT

Protective breathing apparatus incorporates a powered air pump which introduces potentially contaminated air from the atmosphere to the ‘process chamber’. The process chamber are composed of a filter and disinfectant solution such as soap, alcohol by adding the solution mixed with the contaminated air passes through the filter to catch a virus in the inlet air to separate the processed air and solution. Furthermore the processed air can be heated and/or be exposed to UV light, the air is then forced to pass through a disinfectant into a centrifuge. The centrifuge then spins the disinfected air forcing the air through a filter, the air then passes a mechanical filter resulting in purified air that flows through conduit means into a transparent plastic breathing hood containing the wearer's head. The hood is secured by a neck band around the wearer's neck. Newly purified air in the breathing hood is distributed and circulated throughout the hood's interior, the current of air holds the hood out of contact with the wearer's head. After the purified air has been inhaled and exhaled by the wearer, it is expelled through an exhaust port in the breathing hood. The exhaled air has potential to be contaminated. The exhaust air travels through conduit means back to the process chamber, this redirected exhaust air is purified before its release back into the atmosphere so no contaminates are expelled into the atmosphere. Optional provision is made for adjusting the temperature of the newly purified air for optimal user comfort, and for a plurality of breathing hoods to be supplied by a single air purifying device.

The above device can be stationary without the breathing hood. Similar to a dust cleaner, but it can kill the virus.

### BACKGROUND OF THE INVENTION

This Invention is based off of the Protective Breathing Apparatus by inventor of Peter Sing U.S. Pat. No. 4,815,458 which use simple filters to block larger particles such as, dust, pollen, etc. . . . which are not able to block much finer particles such as viruses, like COVID-19, which has a diameter of roughly 120 nanometers (nm). It does not catch the contaminants from user exhale, which will not stop the transmission of the COVID-19 virus. The most distinct difference is, the previous invention by Peter Sing does not kill the virus, or other living contaminants, as this new improved invention by Peter Sing does. This new virus exterminator will use a less dense filter by using disinfectant to kill the virus while processing the air. This new application will improve the previous invention. This new invention will possibly eradicate viruses, especially COVID-19 from the earth. Numberless situations exist in which wholesome air for breathing is not available without some means for removing the contaminants from the atmosphere. This is increasingly prevalent in today's society with the novel ‘Coronavirus’ at large. Normally, conditions for using a protective breathing apparatus are, but not limited to; industrial areas, mines, painting and cleaning operations, etc. . . . . Now, everyone in the world needs to wear a protective breathing apparatus in the form of face masks to enter the public world because the untreated air may contain life-threatening contaminants.

The protective masks available today may stop a virus from being inhaled or exhaled, but if it were to filter out a virus then that virus would be attached to the mask itself and remain alive for an undetermined amount of time. These face masks don't protect your eyes or even kill the virus. By purifying not only the air that is being inhaled, but also purifying the air being exhaled prevents an unknowingly infected person from releasing their contaminants back into the atmosphere, thus, stopping the spread of an infectious disease.

It is a primary object of this invention to provide an effective inexpensive apparatus for supplying purified healthful air for breathing, with protection and comfort for the eyes and face of the user at the same time. It is further an object of this invention to provide an apparatus easily portable and lightweight enough for prolonged wearing without discomfort. It is also an object of this invention to supply simultaneously the heads and faces of multiple users with air that has not only been freed of any harmful gaseous, liquid, solid, or infectious impurities but also adjusted in temperature for maximum user comfort. These and other objects will be explored in detail in the ensuing disclosure.

### SUMMARY OF THE INVENTION

Atmospheric air is drawn into the intake which goes directly into the first fan, compressing and forcing the air through the first filter. The air then passes through a reducer to compress the air once more. The newly compressed and filtered air then passes into another fan but this time the air is combined with the disinfectant at a high rate of speed causing the air and disinfectant to separate through centrifugal forces. The separated disinfectant then drains into a reservoir to be re-used while the separated air is forced through another reducer to a chamber that has optional UV light or added heat. The newly purified air then passes through conduit means into the breathing hood and is inhaled by the wearer. The breathing hood encompasses the wearer's head entirely and is secured by a neck band around the wearer's neck. Newly purified air flows into the breathing hood, this current holds the hood out of contact with the wearer except for where it is secured with the neck band. After the wearer exhales the air it is expelled through an exhaust port which returns the expelled air back to the processing chamber to purify the air once more before releasing the exhausted air back into the atmosphere.

Optional provisions can be made to attach multiple breathing hoods to a single purifier allowing a plurality of breathing hoods to be supplied with clean, uncontaminated air from only a single purifier. Also, the incoming air supply may have temperature control for the user's optimal comfort. The purifier can be separated from the breathing hood allowing the user to attach the purifier to an air conditioning or heat pump unit.

The process chambers will compress the air before or after processing and before or after storing the air. Compressed air can be stored in at least one storage tank that will be either before or after the disinfection processing. Compressing air creates a smaller area needed to process air and disinfectant. Pressurized air and disinfectant forces the two to mix thoroughly. The device will use a small DC powered air pump to compress the air and/or disinfectant. Compressed air will be easier to mix with the disinfectant. Another solution to mix the air and disinfectant is the use of a spherical object inside of a conduit. The air and disinfectant will be forced to pass by the spherical object, forcing the two to collide and combine thoroughly. Another form of disinfection that can be applied is the use of a heating element. The air may be cooled/condensed in order to remove excess moisture after the air is processed. There can be a valve to regulate the pressure reaching the breathing hood.

Further filtration can be introduced by filling the intake conduit with packing materials; like mesh, cotton, plastic pellets, wood pellets, etc. . . . . This further filtration will allow for the air that is introduced to the processing chamber(s) will have already been removed of any larger toxic particulates.

Optional provisions can be made for the device to attach to an auxiliary powered air blower, such as a vacuum. The processor can be added to the motor of any vacuum and disinfect the air as it would with the portable device. There will be a regulator and/or compressor to limit the air flow from such a device.

These and other concepts of this invention will now be described in full detail in connection with the accompanying illustrative drawings, wherein:

### SHORT FIGURE DESCRIPTION OF DRAWINGS

FIG. 1 is a left elevational view of the preferred embodiment of this invention, to diagram the system for providing purified air.

FIG. 2 is a left side elevational view of the preferred embodiment of the breathing hood.

### DESCRIPTION OF THE PREFERRED EMBODIMENTS

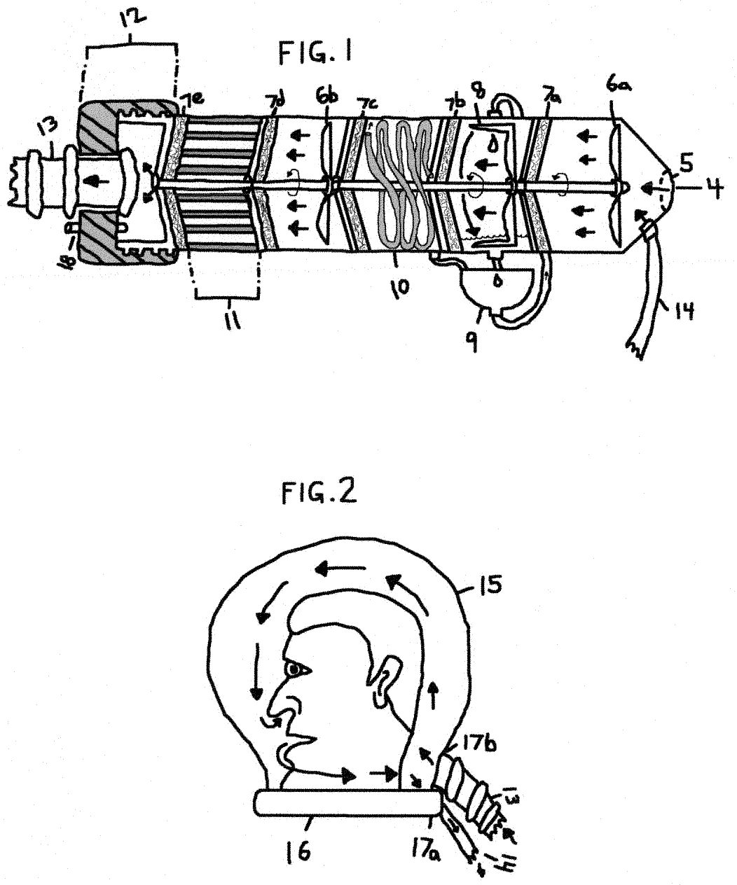

The protective breathing apparatus shown in FIGS. 1-2 uses an electrical motor to draw air from the atmosphere through the intake 4, which has a metallic, or non-metallic, mesh 5 at its threshold, as well as the exhaust air from the breathing hood via conduit 14. Fan 6a aides in drawing in air from the intake 4 but also compresses the air, forcing air to pass through a filter 7a as well as a reducer into the next stage comprised of a fan with angled blades 8 to compress the air once more and to mix the air with the disinfectant (soap, alcohol, etc. . . . ) produced from the disinfectant reservoir 9. The disinfectant reservoir 9 collects the disinfectant after it has been separated from the air through centrifugal forces 8 as well as gravity through the means of distillation 10 and stores disinfectant to be drawn back into the chamber to disinfect the passing air. Once the air is separated by centrifugal forces caused by fan 8 the compressed and separated air passes through another filter 7b and reducer into the distiller 10 to further separate any remaining disinfectant particles. The newly disinfected air passes through another filter 7c and reducer to be compressed by fan 6b and pass through a filter 7d and reducer into the heat/UV light chamber 11 to destroy any and all remaining contaminated particles in the air. The air passes a filter 7e and reducer to be released to the pressure regulation chamber 12 where pressure can be released, if it is too great for the conduit 13 or breathing hood in FIG. 2, through the pressure regulator 18. The freshly purified air then passes through the conduit 13 to the breathing hood in FIG. 2.

Once the purified air reaches the end of the conduit 13 it is then released through the inlet 17b inside of the transparent plastic hood envelope 15 inflating the hood envelope so there is no contact with the wearer's head. The transparent plastic hood envelope 15 is attached to an easily adjustable and removable neck band 16 to secure the breathing hood to the wearer whilst insuring an impregnable seal. The fresh, clean, purified air is now safe to breathe. After the wearer inhales and exhales the cleaned air, the now sullied air is expelled through the exhaust port 17a to be returned through conduit 14 to the intake of the purifying device 4 to be purified once more before being reused in the apparatus or released into the atmosphere. The device is designed to draw in air from the atmosphere that may be polluted or contaminated (with dust, allergens, viruses, infectious disease, etc. . . . ) and create a safe and protected breathing environment for the user as well as anyone around the user. This is because the device only releases purified air. The user could have a very contagious illness or disease and still interact with others in a public setting because the contaminated air they exhale is cleansed before it is released into the atmosphere. This protective breathing apparatus would stop the transmission of any respiratory disease from person to person. This disclosure is intended to be illustrative; various substitutions and combinations of elements lie within the scope of its concepts and structures, which are defined and limited only by the ensuing claims.

While the foregoing written description of the invention enables one of ordinary skill to make and use a product incorporating a composite stiffener as described above, those of ordinary skill will understand and appreciate the existence of variations, combinations, and equivalents of the described embodiments, methods, and examples herein. Thus, the invention as claimed should therefore not be limited by the above described embodiments, methods, and examples, but by all embodiments and methods within the scope and spirit of the claimed invention.

What is claimed is:

1. Protective breathing apparatus to provide clean air free of hazardous, toxic fumes, virus pollutants, and dust in an area where the air may be unsuitable for safe breathing. It protects the user's face and entire head, to avoid contact with toxic material such as virus, toxic fumes, toxic chemicals, etc. . . . It is a protective breathing apparatus without using a heavy full face mask, which is uncomfortable to wear. It consists of blowing or compressing ambient air into the air intake of the apparatus to collect ambient air which may be contaminated by viruses, dusts, and other pollutants. The contaminated air travels to the process chamber which purifies by mixing with a disinfectant (such as soap, alcohol, etc. . . . ) using a similar method to an air scrubber. Furthermore, the mixed air and disinfectant solvent pass through a filter, which will catch the contaminants and break them down or kill them. Furthermore the disinfectant is released into a chamber containing a fan with angled blades that will turn to create centrifugal force that forces air and disinfectant to mix and have more contact to kill virus and remove harmful fumes, dust, etc. . . .

2. Circulation and distribution, in accordance with claim 1; the atmospheric air that went through the first stage in the cleaning process then moves forward to the next stage in the cleaning process. Any remaining disinfectant will be separated and replaced back into the disinfectant reservoir. There is at least one of these stages to filter and mix air with the disinfectant solvent to provide better results of breaking down or killing contaminants.

3. In accordance with claim 1, distribution means that the clean air, after being filtered and processed by centrifuge, will be guided through a conduit into a flexible transparent plastic breathing hood. The air will then be exhausted after the wearer inhales the clean air and exhales. The exhaust port has a conduit connecting the breathing hood back to the air intake to go through the purifying treatment; such as the intake of ambient air. This will recycle the exhaled air of the user, which may potentially contain a virus, infectious disease, or any other harmful contaminants, to avoid releasing any of the user's potential contaminants into the atmosphere so that transmission of such contaminant is not possible.

4. Circulation of disinfectant solvent, in accordance with claim 1, will occur by collecting solvent after treating the ambient air. Collection system will use gravity and/or centrifugal force to collect in the disinfectant reservoir and pump into the next chamber to complete the process again to more effectively treat the contaminated air.

5. Chamber for using heat and/or UV light for further treatment of contaminated air along with claim 1. Either before or after the ambient air enters the disinfectant process, which consists of centrifugal filter treatment, the air will go to an air process chamber to receive further treatments such as UV and heat treatment to further eliminate the virus or other contaminants.

6. In accordance with claim 1, The breathing hood guides the exhaled breath of the user from the exhaust port, through a conduit, to the intake of the apparatus for further purification to avoid releasing any potential contaminants into the atmosphere.

7. Releasing and adjusting the pressure in accordance to claim 1, the valve controlling the pressure of the plastic hood is controlled by the clean air after being processed by the purifying apparatus. It has two outlet control valves; one is released to the atmosphere after the purifying process, the other valve controlling the air pressure is located inside the plastic hood to ensure it is adequately filled with air to provide enough air to breath as well as keeping the hood out of contact with the wearer's head. The breathing hood has only one control valve directed to release into the atmosphere, but should only be used when the wearer is in a secluded place or anywhere there is no potential to contaminate another person's air. Otherwise all the exhaled air from the user will be re-channeled to the air intake and it will go through a further cleaning process before releasing into the atmosphere.

8. Collection of disinfecting solvent by guiding air and disinfectant solution through a coil pipe in the chamber after the centrifugal disinfection treatment, as in claim 1, by cooling the coil pipe which will contain moisture and clean air, the cold temperature surrounding the coil pipe will condense the disinfectant solution to help separate the disinfectant solvent from the air. The disinfectant solvent will flow to the disinfectant reservoir and be be stored for reuse.

9. In accordance with claim 1, the portable apparatus could be designed to be stationary. It will eliminate the breathing hood. All of the purified air that passes through the purifying treatment will be released inside of a building to clean the interior air, eliminating the virus, pollution, or any other harmful contaminants. All of the interior air will go through this protective breathing apparatus, similar to an air scrubber, without opening the window, or using outside cold/hot air. This will save energy in cold winter and hot summer by recycling indoor air through this virus and pollution purifying machine.

10. Protective breathing apparatus in accordance with claim 1, wherein said conduit means is a flexible fluid hose.

11. Protective breathing apparatus, in accordance with claim 1, is powered by DC or AC electrical current to an accompanying motor, which drives the main shaft and everything attached to it.
